SEQUENCE LISTING (3 sequences)

SEQ ID NO: 1 - protein, 17 aa, Homo sapiens
ALLEAGALPNAPNSYGR

SEQ ID NO: 2 - protein, 12 aa, Homo sapiens
EGFLDTLVVLHR

SEQ ID NO: 3 - protein, 12 aa, Homo sapiens
LPVDLAEELGHR

(19) United States
(12) Patent Application Publication
(10) Pub. No.: US 2020/0232987 A1
(43) Pub. Date: Jul. 23, 2020

(54) Proteomic And Genomic Analysis For Colon Cancer Prognosis

(71) Applicant: NantOmics, LLC, Culver City, CA (US)

(72) Inventors: Sarit SCHWARTZ, Rockville, MD (US); Fabiola CECCHI, Potomac, MD (US); Todd HEMBROUGH, Gaithersburg, MD (US); Stephen Charles BENZ, Santa Cruz, CA (US)

(21) Appl. No.: 16/647,581

(22) PCT Filed: Sep. 18, 2018
(86) PCT No.: PCT/US2018/051479

§ 371 Date: Mar. 16, 2020

Related U.S. Application Data
(60) Provisional application No. 62/560,177, filed on Sep. 18, 2017.
(60) Provisional application No. 62/561,496, filed on Sep. 21, 2017.

(51) Int. Cl. G01N 33/574

(52) U.S. Cl. CPC G01N 33/57419; G01N 2333/4739

(57) ABSTRACT

Methods are provided for identifying colon cancer patients whose genome shows either microsatellite stability (MSS) and/or a low tumor mutational burden (TMB) with a good prognosis, irrespective of treatment strategy. Specific protein fragment peptides of the p16 protein are precisely detected and quantitated by SRM-mass spectrometry directly in MSS and/or low TMB colon tumor cells collected from colon tumor tissue that was obtained from a colon cancer patient. The measured p16 levels are compared to p16 reference levels to determine if the MSS and/or low TMB colon cancer patient will have a longer overall survival.

This application claims priority to U.S. Provisional Application No. 62/560,177, filed Sep. 18, 2017, and to U.S. Provisional Application No. 62/561,496, filed Sep. 21, 2017, the contents of which are both hereby incorporated by reference in their entireties. This application also contains a sequence listing submitted electronically via EFS-web, which serves as both the paper copy and the computer readable form (CRF) and consists of a file entitled “SeqListing_3900_0075I”, which was created on Sep. 18, 2018, which is 853 bytes in size, and which also is incorporated by reference in its entirety.

### INTRODUCTION

New and improved methods are provided for determining the probable course and clinical outcome of a cancer patient with colorectal cancer and the prospects of overall recovery and survival from colorectal cancer of the patient. The method provides for measuring the level of the p16 protein in tumor tissue from colorectal cancer patients when the tumor genome from that tissue has an overall stable genome and does not show a high mutation rate. The methods allow a prognosis of the course of disease for colorectal cancer patients irrespective of treatment strategy such as chemotherapy, molecularly-targeted therapy, and/or immunotherapy.

More specifically, a quantitative mass spectrometry-based proteomic assay for the p16 protein is performed on tumor cells procured from patient tumor tissue in cases where the patient tumor cells exhibit high MicroSatellite Stability (MSS), exhibit no MicroSatellite Instability (MSI) and, optionally, also exhibit low overall Tumor Mutation Burden (TMB).

The presence and/or quantitative levels of p16 expression in tumor cells obtained from patient tumor tissue is determined by quantitating a specified fragment peptide derived from the full-length p16 protein in digests prepared from formalin-fixed tissue samples. The presence and/or quantitative level of p16 can be measured using specific fragment peptides as described in PCT/US2016/033776 (filed May 23, 2016), the contents of which is hereby incorporated by reference in its entirety. If specific levels of the specified p16 fragment peptide are found to be below a specified quantitative level in tumor cells present within patient tumor tissue, and the cells also show MSS and/or TMB below a specified level then the patient from which the patient tumor tissue was obtained will have a good prognosis, reflected by a longer overall survival as measured in days.

The specified p16 fragment peptide is detected using mass spectrometry-based Selected Reaction Monitoring (SRM), also referred to as Multiple Reaction Monitoring (MRM), and referred to herein as an SRM assay. An SRM assay is used to detect the presence and quantitatively measure the amount of the specified fragment peptides, directly in cells procured from cancer patient tissue, such as, for example formalin fixed cancer tissue. The amount of the specific peptides is then used to quantitate the amount of intact p16 protein in the tumor sample.

### BRIEF DESCRIPTION OF THE FIGURES

FIG. 1 shows the overall survival curve of 145 colon cancer patients; those patients whose tumor cells demonstrate MSI (Microsatellite Instability) in their genome have longer overall survival as compared to those colon cancer patients whose tumors show MSS (Microsatellite Stability) in their genome, irrespective of administered treatment. Results are statistically significant (p=0.0031).

FIG. 2 shows the overall survival curve of 145 colon cancer patients and demonstrates that those colon patients whose tumor cells show a high level of TMB in their genome have longer overall survival as compared to those colon cancer patients whose tumors show low TMB in their genome. Results are statistically significant (p=0.0181).

FIG. 3 shows the survival curve of 145 colon cancer patients and demonstrates that p16 quantitative SRM levels in tumor cells procured from colon tumor tissue from the patients is only slightly prognostic for predicting which patients will live longer. A p16 quantitative cutoff of 108 amol/μg demonstrate results irrespective of microsatellite status, tumor mutation burden, or administered treatment. Results are only slightly statistically significant (p=0.0351).

FIG. 4 shows the overall survival curve of a cohort of 100 colon cancer patients whose tumors show MSS (Microsatellite Stability) in their genome, which demonstrates that p16 quantitative SRM is able to provide prognostic information about which of the patients will have overall longer survival in this MSS patient population. A p16 quantitative cutoff of 108 amol/μg demonstrates results irrespective of tumor mutation burden or administered treatment. Results are statistically significant (p=0.0021).

FIG. 5 shows the overall survival curve of 106 colon cancer patients whose tumors show low TMB in their genome, which demonstrates that p16 quantitative SRM is able to provide prognostic information about which of the patients will have overall longer survival. A p16 quantitative cutoff of 108 amol/μg demonstrates results irrespective of microsatellite status or administered treatment. Results are statistically significant (p=0.0016).

FIG. 6 shows the overall survival curve of 106 colon cancer patients whose tumors show a combination of low TMB plus MSS (microsatellite stability) in their genome and demonstrates that p16 quantitative SRM is able to provide prognostic information about which of the patients from this patient population will have overall longer survival. A p16 quantitative cutoff of 108 amol/μg demonstrates positive prognostic results irrespective of administered treatment. Results are statistically significant (p=0.0016).

### DETAILED DESCRIPTION

Methods are provided for determining the prognosis of a colon cancer patient by measuring expression of p16 in tumor cells obtained from a tumor tissue sample from the patient, and where the genome in tumor cells from the patient shows microsatellite stability and a low rate of mutations. The tumor sample is advantageously a formalin-fixed sample. Using an SRM assay that measures a specific p16 peptide fragment, and particular characteristics about the peptide, the amount of the p16 protein in cells derived from formalin fixed paraffin embedded (FFPE) tissue is determined. The peptide fragment derived from the full-length p16 protein has the following amino acid sequence: ALLEAGALPNAPNSYGR.

Detection and accurate quantitation of specific peptides from any of the proteins in digests of FFPE tissue is performed via mass spectrometry using the methodology of Selected Reaction Monitoring (SRM). See, for example, U.S. patent application Ser. No. 13/993,045, the contents of which are hereby incorporated by reference in their entirety.

More specifically, the p16 SRM assay can measure this p16-specific peptide directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue® reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water. A small amount of stabilizer, usually methanol, is added to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature.

Results from the SRM assay can be used to correlate accurate and precise quantitative levels of the p16 protein within the specific cancer of the patient from whom the tissue was collected and preserved, including colorectal cancer tissue.

p16, also known as cyclin-dependent kinase inhibitor 2A and multiple tumor suppressor 1 among others, is a tumor suppressor protein that in humans is encoded by the CDKN2A gene. p16 plays an important role in cell cycle regulation by decelerating cells progression from G1 phase to S phase, and therefore acts as a tumor suppressor that is implicated in the prevention of cancers, notably melanoma, oropharyngeal squamous cell carcinoma, cervical cancer, and esophageal cancer. The CDKN2A gene is frequently mutated or deleted in a wide variety of tumors. p16 is also a known inhibitor of cyclin dependent kinases such as CDK4 and CDK6 which can eventually result in progression from G1 phase to S phase thus promoting tumor cell growth.

The most widely-used methodology presently applied to determine protein presence in cancer patient tissue, especially FFPE tissue, is immunohistochemistry (IHC). IHC methodology uses an antibody to detect the protein of interest. The results of an IHC test are most often interpreted by a pathologist or histotechnologist. This interpretation is subjective and does not provide quantitative data that are predictive of sensitivity to therapeutic agents that target specific oncoprotein targets. Thus, an IHC test cannot determine whether or not a colorectal cancer patient will have a good or bad prognosis when their tumor genome exhibits MSS or low TMB levels.

Studies involving other IHC assays, such as the Her2 IHC test, suggest the results obtained from such tests may be wrong or misleading. This is probably because different laboratories use different rules for classifying positive and negative IHC status. Each pathologist running a test also may use different criteria to decide whether the results are positive or negative. In most cases, this happens when the test results are borderline, i.e. the results are neither strongly positive nor strongly negative. In other cases, tissue from one area of cancer tissue can test positive while tissue from a different area of the cancer tests negative.

Inaccurate IHC test results may mean that patients diagnosed with cancer do not receive the best possible care. If all or part of a cancer is positive for a specific target oncoprotein but test results classify it as negative, physicians are unlikely to implement the correct therapeutic treatment, even though the patient could potentially benefit from agents that target the oncoprotein. If a cancer is oncoprotein target negative but test results classify it as positive, physicians may use a specific therapeutic treatment, even though the patient is not only unlikely to receive any benefit but also is exposed to the agent's secondary risks. In addition, as in the case of prognostic indicators of cancer survival, obtaining wrong protein expression analysis through IHC can give an attending physician inaccurate information about the likely course of treatment outcome.

Thus there is great clinical value in the ability to correctly evaluate quantitative levels of the p16 protein in tumors, especially colorectal tumors that exhibit MSS and low TMB levels, so that the patient will have the greatest level of confidence in his/her likely course of disease and survival outcome.

Detecting and quantitating a specific p16 fragment peptide is performed in a mass spectrometer by the SRM methodology, in which the SRM signature chromatographic peak area of each peptide is determined within a complex peptide mixture present in a liquid tissue lysate (see U.S. Pat. No. 7,473,532, as described above). Quantitative levels of the p16 protein is then determined by the SRM methodology whereby the SRM signature chromatographic peak area of an individual specified peptide from the p16 protein in one biological sample is compared to the SRM/MRM signature chromatographic peak area of a known amount of a “spiked” internal standard for each of the individual specified fragment peptides.

In one embodiment, the internal standard is a synthetic version of the same fragment peptide where the synthetic peptide contains one or more amino acid residues labeled with one or more heavy isotopes, such as ²H, ¹⁸O, ¹⁷O, ¹⁵N, ¹³C, or combinations thereof. Such isotope labeled internal standards are synthesized so that mass spectrometry analysis generates a predictable and consistent SRM signature chromatographic peak that is different and distinct from the native fragment peptide chromatographic signature peaks and which can be used as comparator peaks. Thus when the internal standard is “spiked” in known amounts into a protein or peptide preparation from a biological sample and analyzed by mass spectrometry, the SRM signature chromatographic peak area of the native peptide is compared to the SRM signature chromatographic peak area of the internal standard peptide, and this numerical comparison indicates either the absolute molarity and/or absolute weight of the native peptide present in the original protein preparation from the biological sample. Quantitative data for a fragment peptide is displayed according to the amount of protein analyzed per sample.

In order to develop the SRM assay for the fragment peptides additional information beyond simply the peptide sequence needs to be utilized by the mass spectrometer. This additional information is used to direct and instruct the mass spectrometer, (e.g., a triple quadrupole mass spectrometer) to perform the correct and focused analysis of the specified fragment peptides. An SRM assay may be effectively performed on a triple quadrupole mass spectrometer. That type of a mass spectrometer may be considered to be one of the most suitable instruments for analyzing a single isolated target peptide within a very complex protein lysate that may consist of hundreds of thousands to millions of individual peptides from all the proteins contained within a cell. The additional information provides the mass spectrometer, such as a triple quadrupole mass spectrometer, with the correct directives to allow analysis of a single isolated target peptide within a very complex protein lysate. SRM assays also can be developed and performed on other types of mass spectrometer, including MALDI, ion trap, ion trap/quadrupole hybrid, or triple quadrupole instruments, but presently the most advantageous instrument platform for SRM assay is often considered to be a triple quadrupole instrument platform. The additional information about target peptides in general, and in particular about the specified fragment peptides, may include one or more of the mono isotopic mass of each peptide, its precursor charge state, the precursor m/z value, the m/z transition ions, and the ion type of each transition ion. The peptide sequence of the specified p16 fragment peptide and the necessary additional information as described for the specified fragment peptide is shown in Table 1.

TABLE 1

| | | | Pre- | | | |
|---|---|---|---|---|---|---|
| | | Mono | cursor | Pre- | Tran- | |
| | Peptide | Isotopic | Charge | cursor | sition | Ion |
| SEQ ID | sequence | Mass | State | m/z | m/z | Type |
| SEQ ID | ALLEAGALP | 1712.8845 | 2 | 857.45 | 626.35 | b6 |
| NO: 1 | NAPNSYGR | | | | | |
| | | | 2 | 857.45 | 693.331 | y6 |
| | | | 2 | 857.45 | 975.464 | y9 |

The described SRM p16 assay is performed on tumor cells procured from patient tumor tissue that exhibits MSS and/or low TMB levels, especially in tumor tissue from colon cancer patients. Determining if patient tumor cells show MSI or MSS is performed via whole genome sequencing of the DNA obtained from the patient tumor tissue whereby the genomic sequence from the DNA is compared to the whole genome sequence of the DNA obtained from normal blood cells from the patient. In this way the normal status of the cancer patient DNA is used as a benchmark against which the stability or instability of specific microsatellite regions within the standard genome have been either rearranged, deleted, or duplicated in the tumor genome, indicating MSI, or do not rearrange, delete, or duplicate and thus remaining stable within the tumor genome, indicating MSS.

Similarly, determining the overall mutation rate of the tumor genome is performed via whole genome sequencing of the DNA obtained from the patient tumor tissue whereby the genomic sequence from the DNA is compared to the whole genome sequence of the DNA obtained from normal blood cells from the patient. In this way the normal status of the cancer patient DNA is used as a benchmark against which the total number of mutations are determined thus allowing for generation of a Tumor Mutation Burden (TMB) index which is reflected as the number of mutations per megabase of tumor genome.

To determine an appropriate reference level for p16 protein quantitation, tumor samples were obtained from a cohort of patients suffering from cancer, in this case colon cancer. The colon tumor samples were formalin-fixed using standard methods and the level of p16 in the samples was measured using the methods as described above. The total number of patients in this cohort for this study was 145 (n=145). Patients in the cohort were treated with a variety of therapeutic strategies and thus no correlation was made to specific treatment outcome but to the prognostic condition of overall survival independent of treatment strategies. Clinical response of the patients in this case was measured using methods well known in the art, for example by recording the overall survival (OS) of the patients at time intervals after treatment. A suitable reference level was determined using statistical methods that are well known in the art, for example by determining the lowest p value of a log rank test. Once a reference level was determined it was used to correlate with those patients whose genomes exhibited MSS and/or specific levels of TMB. p16 protein expression levels below the specified reference level of 108 amol/μg protein analyzed in those tumor samples that exhibit MSS and low TMB levels indicated that such patients have a better prognosis vs. those patients whose tumors exhibit MSS and/or low levels of TMB and show p16 protein expression above the specified reference level of 108 amol/μg, irrespective of treatment strategy, as measured by extending the life (survival) of the patient in days.

The presence of MSI and high levels of TMB in tumors, especially colon tumors, are known to be good prognostic determinants for colon cancer, indicating a high likelihood that a cancer patient whose tumor genome shows MSI and high TMB will respond favorably to therapeutic treatment, irrespective of the wide variety of treatment strategies and therapeutics that may be administered to the patient. However, it is also well known in the art that it is difficult to predict the therapeutic outcome for colon cancer patients whose tumors exhibit MSS and low TMB levels—some patients have a good prognosis while others do not. There has been no method by which these two populations of MSS and/or low TMB patients can be subdivided between those with good prognosis (longer survival) and those with bad prognosis (shorter survival). The presently described methods provide for the first time the ability to determine which patients whose tumors exhibit MSS and/or low TMB levels demonstrate good prognosis and will likely survive longer as measured by days.

### Example: Determining Prognosis of Colon Cancer Patients Using the Predictive Value of p16 Protein Expression Levels in Tumor Cells from Patients Whose Tumor Genome Exhibits MSS and Low TMB

### Patients

145 patients were identified with colorectal cancer (CRC). Tumors were surgically removed prior to treatment and archived as formalin-fixed, paraffin-embedded (FFPE) tissue and all were histologically confirmed as CRC. Some patients were treated with a variety of therapeutic agents such as chemotherapy and/or immunotherapy drugs while some were not treated with any therapeutic drug.

### Methods

Tumor cells from FFPE tumor tissue were procured and isolated from the tumor tissue by tissue microdissection and solubilized for downstream mass spectrometry analysis using the Liquid Tissue reagents as described above. Protein levels were quantitated for 29 different proteins using selected reaction monitoring mass spectrometry (SRM-MS), including protein levels for the p16 protein. Overall survival curves of the patients in this study as related to levels of the p16 protein were developed. The microsatellite status and tumor mutation status were determined for many tumors by collecting tumor cells from the FFPE tumor tissue and preparing purified DNA lysates using standard methods well known in the art followed by whole genome sequencing. Blood from the patients was collected whereby purified DNA lysates were prepared from the normal lymphocytes present within patient blood followed by whole genome sequencing. Microsatellite status and tumor mutation burden for each tumor tissue sample was determined by comparing the tumor genome to the normal genome obtained from the blood-borne cells.

### Results

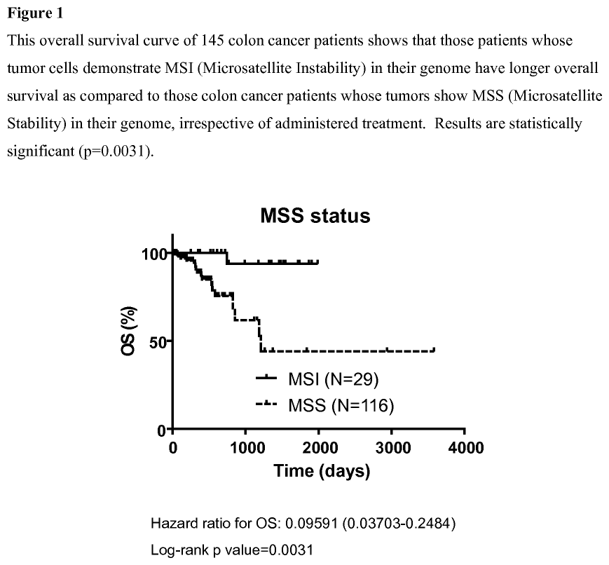

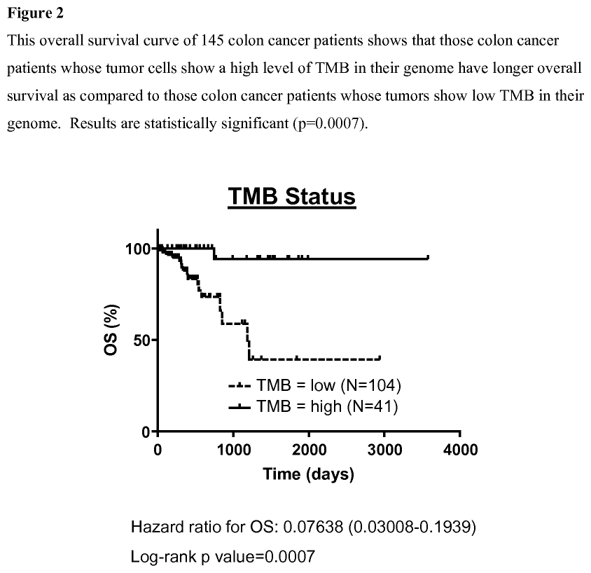

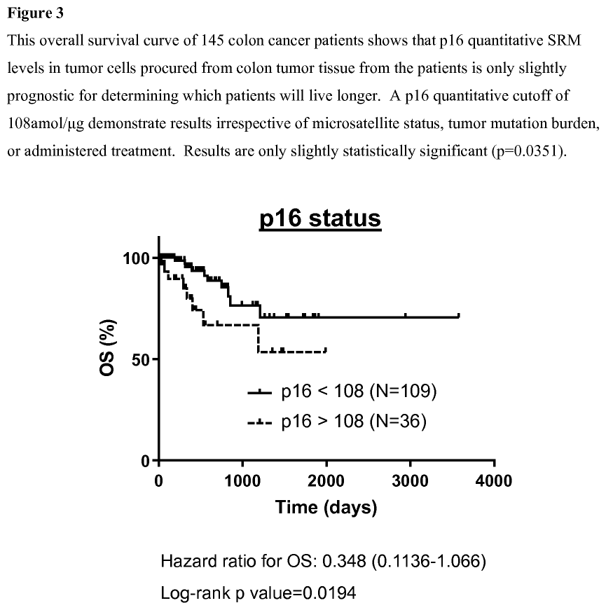

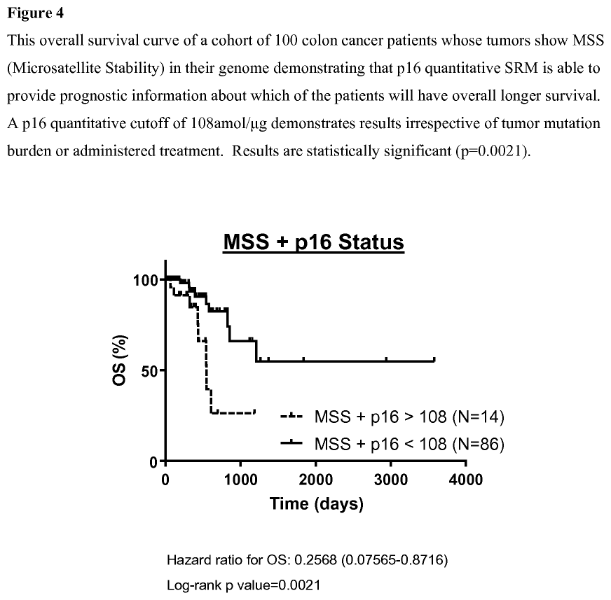

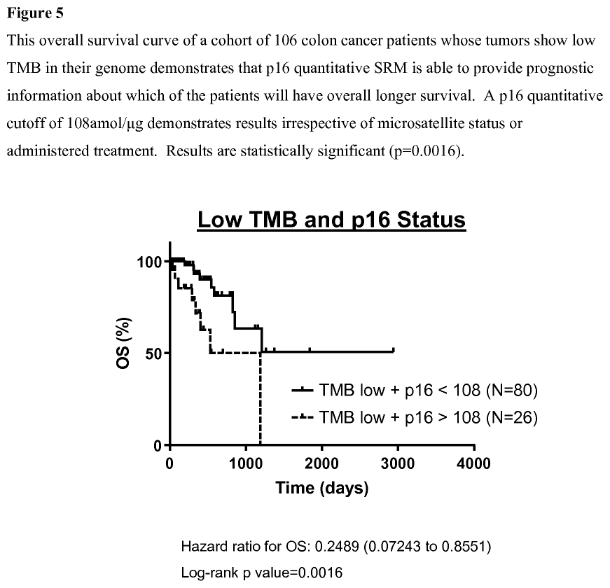

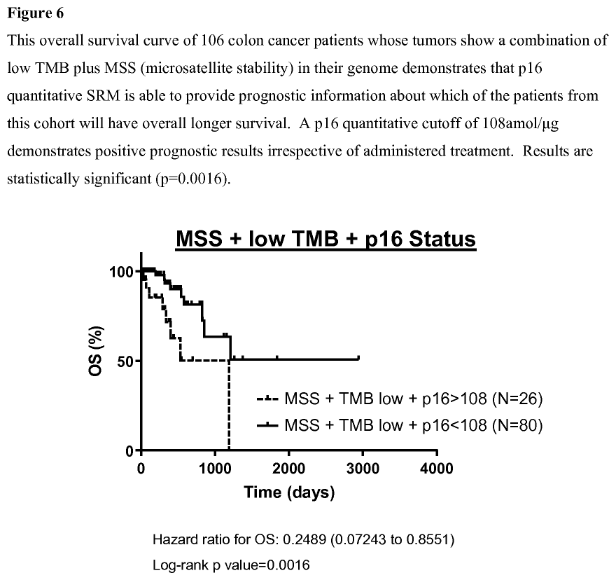

Quantitative SRM data across a number of proteins including proteins specifically targeted by the therapeutic drugs showed no correlation with prognosis value in this cohort of colon cancer patients, except for the p16 protein. A positive tumor genome MSI status (positive for microsatellite instability) analyzed across 146 patients was highly correlated with good prognosis irrespective of the administered treatment, while low TMB (low mutation rate) in the tumor genome also correlated with good prognosis, again irrespective of administered treatment. There were no samples found with MSI/low TMB and no samples found with MSS/high TMB. Focusing on p16 levels indicated that a p16 SRM assay quantitative cutoff of 108 amol/μg on samples showing MSS and/or low TMB showed a statistically significant ability to predict a good prognosis for this particular subset of this population of colon cancer patients. Data from the analyses are shown in FIGS. 1-6.

1. A method for determining the prognosis of a colon cancer patient exhibiting microsatellite stability (MSS) and/or low tumor mutational burden (TMB) comprising:
(a) detecting and quantifying a level of a p16 fragment peptide in a protein digest prepared from a colon cancer tumor sample obtained from said colon cancer patient by selected reaction monitoring using mass spectrometry;
(b) comparing the level of said p16 fragment peptide to a reference level, and
(c) determining the prognosis of said colon cancer patient when the level of the p16 fragment peptide is above or below said reference level.

2. The method according to claim 1, wherein said patient suffering from colon cancer exhibits microsatellite stability (MSS).

3. The method according to claim 1, wherein said patient suffering from colon cancer exhibits low tumor mutational burden (TMB).

4. The method according to claim 1, wherein said patient suffering from colon cancer exhibits microsatellite stability (MSS) and low tumor mutational burden (TMB).

5. The method according to claim 1, wherein said reference level is 108 amol/μg, +/−250 amol/μg, +/−200 amol/μg, +/−150 amol/μg, +/−100 amol/μg, +/−50 amol/μg, or +/−1 amol/μg of tumor sample protein analyzed.

6.-11. (canceled)

12. The method according to claim 1, wherein said protein digest comprises a pro-tease digest.

13. The method according to claim 12, wherein said protease digest comprises a trypsin digest.

14. The method according to claim 1, further comprising the step of fractionating said protein digest prior to detecting and/or quantifying the amount of said fragment peptide.

15. The method according to claim 1, wherein the mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

16. The method according to claim 15, wherein a mode of mass spectrometry used is Selected Reaction Monitoring (SRM), Multiple Reaction Monitoring (MRM), Parallel Re-action Monitoring (PRM), intelligent Selected Reaction Monitoring (iSRM), and/or multiple Selected Reaction Monitoring (mSRM).

17. The method according to claim 1, wherein quantifying the p16 fragment peptide comprises determining the level of the p16 fragment peptide in the tumor sample by comparison to an added internal standard peptide of known amount.

18. The method according to claim 17, wherein the internal standard peptide is an isotopically labeled peptide.

19. The method according to claim 18, wherein the isotopically labeled internal standard peptide comprises one or more heavy stable isotopes selected from ¹⁸O, ¹⁷O, ³⁴S, ¹⁵N, ¹³C, ²H and a combination thereof.

20. The method of claim 1, wherein the p16 peptide has an amino acid sequence as set forth in SEQ ID NO:1.

21. The method according to claim 1, wherein the tumor sample is a cell, collection of cells, or a solid tissue.

22. The method according to claim 20, wherein the tumor sample is formalin fixed solid tissue.

23. The method according to claim 21, wherein the tissue is paraffin embedded tissue.

24. The method according to claim 1, furthering comprising determining the presence or absence of microsatellite instability (MSI) by genomic DNA sequencing.

25. The method according to claim 1, further comprising determining the presence, absence, or a level of the TMB by genomic DNA sequencing.

26. The method according to claim 1, wherein detecting and quantifying the p16 fragment peptide is combined with detecting and quantifying other peptides from other proteins in multiplex.

27. The method according to claim 1, wherein when said level of said p16 fragment peptide is lower than said reference level, then said prognosis is a prediction that said patient will survive longer as measured in days than if said level of said p16 fragment peptide is higher than said reference level.

28. The method according to claim 1, wherein when said level of said p16 fragment peptide is higher than said reference level, then said prognosis is a prediction that said patient will not survive longer as measured in days than if said level of said p16 fragment peptide is lower than said reference level.
